(19) United States
(12) Patent Application Publication
(10) Pub. No.: US 2025/0194977 A1
(43) Pub. Date: Jun. 19, 2025

(54) Methods, Systems, and Media for Creating a Virtual Emotional Social Media Support Network

(71) Applicant: Max B. Mellman, Beverly Hills, CA (US)

(72) Inventor: Max B. Mellman, Beverly Hills, CA (US)

(21) Appl. No.: 18/540,675

(22) Filed: Dec. 14, 2023

(51) Int. Cl. A61B 5/16; G06Q 50/00; H04L 67/306

(52) U.S. Cl. CPC A61B 5/165; G06Q 50/01; H04L 67/306

(57) ABSTRACT

A method, system, and media for creating a virtual emotional social media support network includes a server processor programmed to receive information associated with a first user of a first user device from a plurality of data sources. The data sources include the user device, having a display mechanism and an input mechanism. The data sources also including a user profile associated with the first user and a user-specific database associated with the first user. The server identifying and optimizing a type and quantity of emotionally supportive information configured to support the emotional health of a second user based on information associated with the first user, and providing an algorithm accessible by the server processor configured to determine when the second user may need emotional support in conjunction with the received information associated with the first user device of the first user.

### FIELD OF THE INVENTION

This application relates to methods, systems, and media for creating a virtual emotional support network. In some implementations, the method includes: receiving information associated with a user of a user device from multiple data sources, where the user device comprises a display/input method; identifying an optimized way to support the emotional health of the user based on the received information associated with the user; determining when the user may need emotional support; and causing the optimal way to support the emotional health of the user to be presented on the user device and/or on the device of one or more user friends, thus creating a virtual emotional support network.

### BACKGROUND

In the field of mental health, there are currently several issues. These include, but are not limited to, access, stigma, shortage of mental health professionals, and the rise of mental health disorders. Disparities in accessing mental health services persist globally, with limited availability, affordability, and geographical barriers being key challenges. Furthermore, many individuals still face discrimination and judgment, leading to reluctance in discussing their mental health concerns openly. In addition, there is a shortage of mental health professionals in many regions, particularly in under-resourced areas. This shortage contributes to long wait times for appointments and inadequate mental health support. Unfortunately, the prevalence of mental health disorders, including depression, anxiety, bipolar disorder, and post-traumatic stress disorder (PTSD), continues to rise worldwide. The factors contributing to this increase are complex and multifaceted.

More specifically, the mental health of young people who are currently using social media is a growing concern. Issues such as bullying, academic pressure, social media use, and identity struggles can impact their well-being. Early intervention and accessible support systems are crucial for addressing these challenges. Also, work-related stress, burnout, and poor mental health in the workplace have gained attention. High-pressure environments, long working hours, and inadequate support structures challenge the well-being of employees. Suicide rates remain a major concern in many countries. The reasons for suicide are complex and can be influenced by multiple factors, including mental health conditions, societal pressures, access to lethal means, and inadequate support systems.

The methods, systems, and media of the present disclosure provide a solution to the above problems by providing users a virtual support network wherein the user can engage in traditional social media while supporting their closest friends and loved ones and receiving emotional support when needed. First, information regarding a user's emotional data and friends are collected and stored. Then, the system analyzes the user's emotional data with the other information associated with the user to suggest to the user and/or user's friends: (1) actions to bolster the user's positive emotions; (2) actions to reduce the user's negative emotions; (3) resources for mindfulness and overall well-being; (4) a traditional social media platform that provides positive content to enhance morale; and (5) a user can train their feed to enhance their mood through additional use. Through being connected via this virtual social-emotional support system, a user's loved ones and the community can boost a user's morale and provide emotional support in times of need without the user explicitly stating that they are going through a hard time or are feeling lonely. This will enhance a user's morale and help prevent and reduce suicide rates, anger, gun violence, theft, depression, etc.

### SUMMARY

In accordance with some implementations of the disclosed subject matter, methods, systems, and media for creating a social media application with an emotional support network are provided.

In accordance with some implementations of the disclosed subject matter, a method for creating a virtual social-emotional support network is provided, the method comprising: receiving information associated with a user of a user device from multiple data sources, where the user device comprises a display/input method; identifying the optimal way to support the emotional health of the user based on the received information associated with the user; determining when the user may need emotional support; and causing the optimal way to support the emotional health of the user to be presented on the user device and/or on the device of one or more user friends.

In accordance with some implementations of the disclosed subject matter, a system for creating a virtual social-emotional support network is provided, the system comprising: a hardware processor that is programmed to: receive information associated with a user of a user device from multiple data sources, where the user device comprises a display/input method; identify the optimal way to support the emotional health of the user based on the received information associated with the user; determine when the user may need emotional support; and cause the optimal way to support the emotional health of the user to be presented on the user device and/or on the device of one or more user friends.

In accordance with some implementations of the disclosed subject matter, a non-transitory computer-readable medium containing computer executable instructions that, when executed by a processor, cause the processor to perform a method for creating a virtual social-emotional support network, the method comprising: receiving information associated with a user of a user device from multiple data sources, where the user device comprises a display/input method; identifying the optimal way to support the emotional health of the user based on the received information associated with the user; determining when the user may need emotional support; and causing the optimal way to support the emotional health of the user to be presented on the user device and/or on the device of one or more user friends.

A user's emotional profile is configured based on an initial setup procedure, wherein a user's individual characteristics are input. Next, the user adds the contact information of their closest friends and family members and sets up under what conditions and how much of the user's emotional-state data is shared with the user's friends. The added friends then accept or decline the invitation to be added to a user's social-emotional support network and can also set up under what conditions and how much of the user's emotional-state data is shared with the friend. This information is transmitted to a server and then stored in a user-profiles database in which each user has a unique profile that is constantly updated with every instance of informational input, e.g., the user's self-reported emotional state.

The system takes every instance of information input to continuously re-evaluate and refine the user's unique emotional and personality profile based on an algorithm. In some embodiments, the system employs artificial intelligence (AI). In AI-based implementations, the AI continuously downloads the latest scientific studies regarding emotional well-being, personality types, etc. and relays relevant data to the server so that the algorithm itself is continuously evolving to better tailor its messages and suggestions to each user.

To establish a baseline emotional state, the system asks the user to self-report their current emotional state through a number system 1-10 on energy levels and 1-10 on pleasant to unpleasant level. The system will analyze these self-reported emotional states to find patterns and correlations with other pieces of information obtained from various data sources which may include the amount of sleep or exercise the user has gotten, local weather conditions, and the amount of work or socializing the user has done. Furthermore, the user may have the option to take various online personality tests to determine traits such as extroversion/introversion, their love language, the way an individual would like to be supported (support test), etc. The system will take the gathered information and analyze it together with the user's self-reported emotional states to provide the user with suggested actions to improve, maintain, or enhance their current emotional state and/or if the user and user's friends' settings allow for it, send reports to user's friends and/or suggestions to user's friends for reaching out to the user when the user may be in need of emotional support. In addition, the system will curate the most appropriate positive content via social media feed, mindfulness, and meditation tools for the user and suggest them.

Upon the system establishing that a user is in need of support, the system will notify their loved ones with a “Send Love” feature. This feature will take input from a user's support test and current friend network to discreetly ping the appropriate person to send the notification to.

Thus, through this social-virtual support system, a user can view positive content, record and monitor their own mental health, take steps to make improvements based on the system's suggestions, increase their own self-awareness through personality tests, mindfulness, meditation and/or counseling, and remain connected to loved ones. In turn, the user's loved ones can rest assured that the user's emotional state is at an acceptable level, or if it is not that they will be notified and will have a chance to reach out to offer support as needed.

### BRIEF DESCRIPTION OF THE DRAWINGS

Note: In the following figures, the name “Feel” is used to reference the methods, systems, and media for creating a virtual emotional support network that is the subject of the present application.

FIG. 1 illustrates an initial setup diagram enabling a user to launch a Feel account and profile of an emotional support network peer-to-peer application.

FIG. 2 illustrates a user interface of the Feel emotional support network application featuring a friend list and pending friend requests.

FIG. 3 illustrates the user interface featuring a mood icon and metrics sliders for indicating a user's mood.

FIG. 4 illustrates the user interface featuring a mood refining tool for selecting specific moods.

FIG. 5 illustrates the user interface featuring a user profile screen with a pop-up message and search icon displayed thereon.

FIG. 6 illustrates the user interface featuring an outgoing message to another user, including predetermined messages querying the user's mental state.

FIG. 7 illustrates the user interface featuring functionality for sending an outgoing video message.

FIG. 8 illustrates the user interface with a predetermined outgoing message selected for sending to another user.

FIG. 9 illustrates the user interface with the mood icon and metrics sliders set in response to the outgoing message.

FIG. 10 illustrates the mood refining tool with multiple moods selected in preparing to respond to the outgoing message.

FIG. 11 illustrates the mood refining tool with a particular mood and intensity selected in preparing to respond to the outgoing message.

FIG. 12 illustrates the user interface featuring a first profile screen identifying a user of the application and biographical information.

FIG. 13 illustrates the user interface featuring a second profile screen identifying posts made by a user of the application.

FIG. 15 illustrates the user interface with a single sensitivity adjustment for managing support sensitivity.

### DESCRIPTION

The present invention is described more fully hereinafter, but not all embodiments are shown. While the invention has been described with reference to exemplary embodiments, it will be understood by those skilled in the art that various changes may be made, and equivalents may be substituted for elements thereof without departing from the scope of the disclosure. In addition, many modifications may be made to adapt a particular structure or material to the teachings of the disclosure without departing from the essential scope thereof.

The drawings accompanying the application are for illustrative purposes only, and are not intended to limit the embodiments of the present application. Additionally, the drawings are not drawn to scale. Common elements between different figures may retain the same numerical designation.

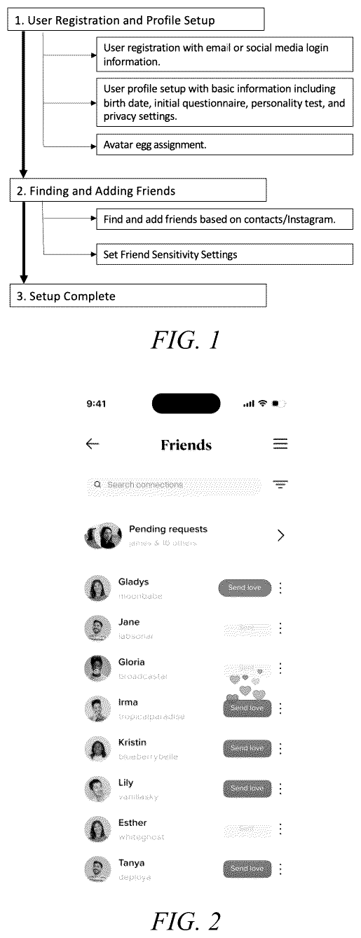

FIG. 1 illustrates a flow diagram of an initial user registration and profile setup of the Feel emotional support application. In step one 10, a user selects a command for setting up a new profile, and proceeds through a series of registration prompts. Registration may include providing the user's email, social media profile information, or other identifying information on which user accounts are customarily based. In some embodiments, a user profile setup may include birthdate or other identifying information on which security prompts may be based. In other embodiments, the user may include a personal photo or a pre-programmed Feel avatar. During profile setup, the user also indicates various privacy settings, which will govern the ability of other users to view the user's biographical information. As part of the initial setup, the user may be prompted with an initial questionnaire, which may include a personality test, thereby establishing a baseline personality type for the user account.

Still referring to FIG. 1, after registration is complete, the user proceeds to step two 12 populating a “friends” field for offering and receiving mental health support. As part of the friend finding process, the application may connect the user with other users as friends based on telephone contacts, or based on social media connections, which include friend identifiers. Once a collection of friends is selected, the user may select sensitivity settings, which govern how often, or under which circumstances the algorithm will push support requests to and from the user. Once the user profile is generated with identifying information as described above, and an initial selection of friends is populated, and sensitivity settings defined, the profile setup process is complete (step three 14).

Referring to FIG. 2, after setup is complete, a user may continue to add friends to the user's network. The application provides a user interface “friends” screen 16 providing a list the user's existing friends 18, a search function 20 for finding other users, as well as access to a pending friend requests page 22. The existing friends contacts each include a status field 24 indicating the contact's desire for supporting contact by the user account, or an indication that contact was previously been made and is not currently requested.

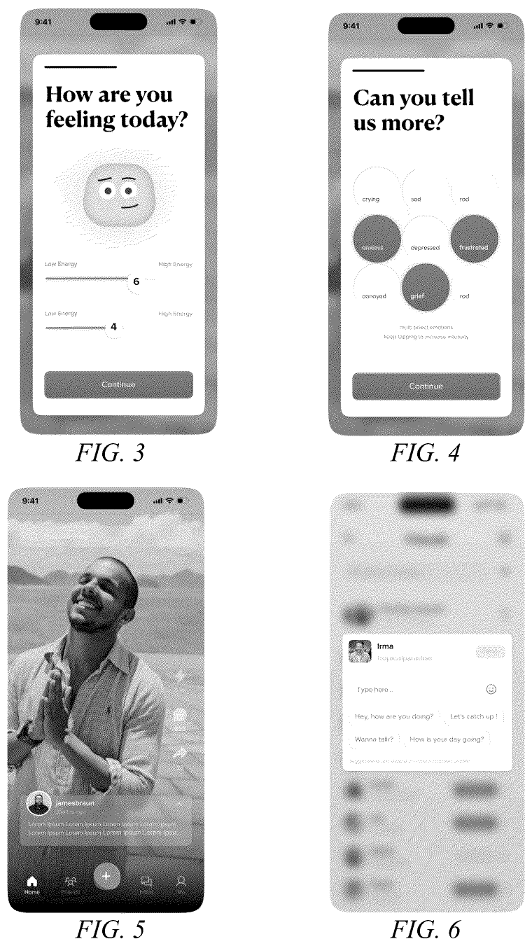

Referring to FIG. 3, the user is prompted vie the application user interface to indicate the user's mood. A mode face icon 26 is provided along with the prompt “How are you feeling today.” In alternative embodiments, another prompt may be displayed. Below the mode face icon 26, an upper “energy” slider 28 and a lower “energy” slider 30 are provided for setting the user's current mood. Once the upper “energy” sider 28 and the lower “energy” slider 30 are set, the user can continue to the next set of prompts.

Referring to FIG. 4, following the initial mood indication, the application prompts the user for more information using a “Can you tell us more?” query 32. prompt. Below the prompt, the user may select among several predetermined emotion buttons 34 which are designed to reflect various emotions, typically in the range of negative emotions. In the illustrated embodiment, a user has selected anxious, frustrated, and grief. In a preferred embodiment, the buttons may be selected multiple times, with the color of the button changing in intensity to allow the user to indicate feeling more of one emotion and less of another. After selecting a range and intensity of emotions to refine the energy readings set in the interface in FIG. 3, the user may continue to request an intervention from the user's predetermine list of “friends.”

Referring to FIGS. 5, a typical user profile screen 40 is shown with an incoming message 42 displayed at the bottom of the screen. The user profile screen 40 includes a “home” icon 44 for returning to the user profile screen from other pages of the user interface, a “friends” icon 46 for moving to the “friends” screen 16 (FIG. 2), a post icon 48 comprising a button labeled with a plus sign for posting content to the social network, an “inbox” icon 50 for accessing messages from other users, and a “me” icon 52, which enables the user to reach controls regarding the user's profile and make changes thereto. In addition to these administrative functions, the user profile screen 40 includes an energy meter icon 54 illustrating the number of connections the user has made to other user accounts, a messages icon 56 which allows the user to access existing conversations with other users and quickly offer more support to user's in emotional distress, and a sent icon 58, which allows the user to access messages that have been sent to other users. Finally, the user profile screen 40 includes a user search bar 60 enabling the user to locate new “friends” using search terms, user account names, or other identifying data.

Referring to FIG. 6, an inquiry prompt 62 is shown. The inquiry prompt 62 is shown overlaid over the “Friends” screen 16, which has been blurred out. The inquiry prompt 62 preferably appears in response to a user selecting a “send love” adjacent an existing friend 18 (FIG. 2) in the “friends” screen 16. The inquiry prompt 62 preferably includes a thumbnail profile photo 64 of the sending user, along with the sending user's profile name 66. A series of pre-populated prompts 68 designed to elicit a response corresponding to the receiving user's current mood are provided, along with a query field 70, which allows the user to compose a personalized message to the recipient. Once the inquiry prompt is sent, the “friends” screen reappears, with a “sent” indicator appearing next to the existing friend 18. See, FIG. 2.

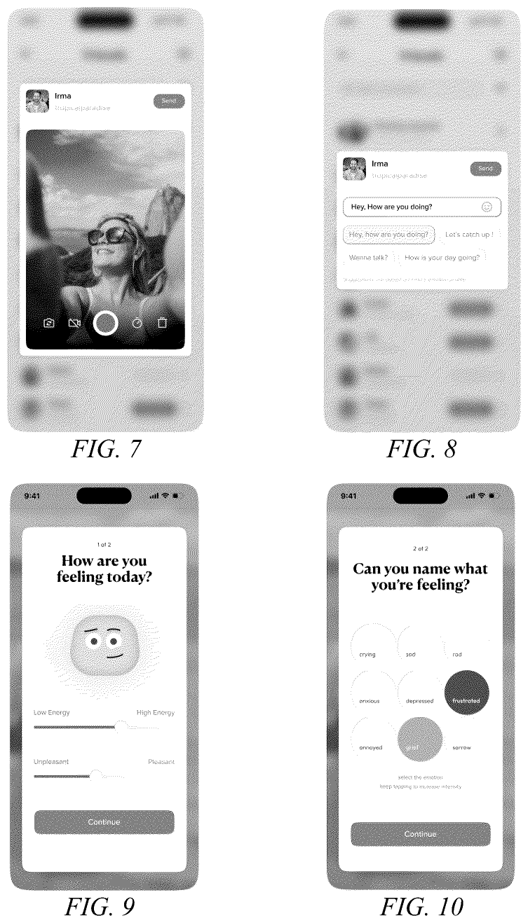

Referring to FIG. 7, in addition to sending predetermined content and customized text, the application allows users to send personalized video content through the application to another user to confer emotional support to the user. By selecting a camera icon 72 in the query field 70 (FIG. 6), a video prompt 74 is shown. The video prompt 74 includes the customary controls one would find on a video camera interface, including a camera reverse icon 76 for reversing the camera view, an audio-only icon, a timer 80 for preparing a timed recording, and a delete icon 82 for discarding recorded video pending re-recording. A user can send the video to the selected user after recording is complete.

Referring to FIG. 8, the inquiry prompt 62 is shown with one of the pre-populated prompts 68 selected prior to sending. In the illustrated embodiment, selecting a pre-populated prompt 68 moves it into the query field 70, thereby allowing the user to add text to the pre-populated prompt 68, or modify it according to preference prior to sending it to a target user.

Referring to FIG. 9, the mood indication interface (FIG. 3) is shown with the mood face icon 26 reflecting the settings on the upper “energy” slider 28 and the lower “energy” slider 30. In the illustrated view, the upper “energy” slider 28 indicates a metric simply of the user's energy, in terms of the intensity of the user's mood. The lower “energy” slider indicates the positive emotion vs. negative emotion nature of the user's current mood. In the illustrated embodiment, the user has indicated a somewhat high energy, and an almost neutral, although slightly negative mood and the mood face icon 26 has reacted to indicate these settings. Once the upper and lower “energy” sliders 28, 30 are set, the user can press continue to refine the emotion settings.

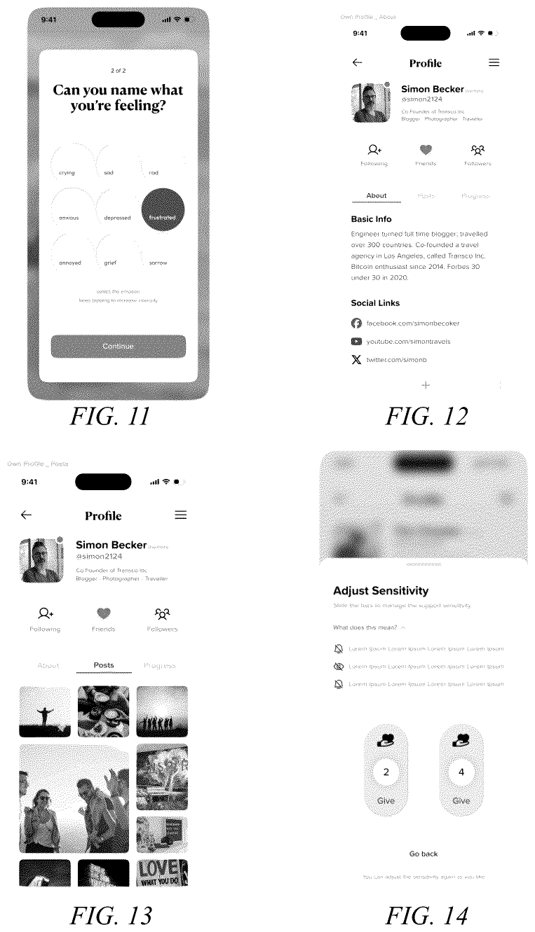

Referring to FIGS. 10 and 11, the “Can you tell me more?” prompt 32 is shown with two of the emotion buttons 34 selected at various degrees of intensity. In the illustrated view of FIG. 10, the “grief” emotion has been selected along with the “frustrated” emotion. By pressing the “frustrated” emotion button multiple times, the user has indicated that as a more intense emotion than the “grief” emotion. The multiple presses of the “frustrated” emotion are reflected in the user interface by the emotion button 24 darkening relative to other selected emotion buttons 24. In the illustrated view of FIG. 11, the “grief” button has been pressed several times until it has cycled around to a white background, as it is initially shown, corresponding to an absence of that emotion. Thus, any of the emotion buttons 34 can be pressed multiple times to reflect varying degrees of those emotions, including an absence of them.

Referring to FIG. 12, an editable user profile page 84 of a user is shown. The editable user profile page 84 is divided into an “about” section 86 as shown, a “posts” section 88, and a “progress” section 90. In the “about” section 86, The user's basic information 92 is shown, comprising a brief biography of the user, preferably authored by the user. In addition to the user's basic information 92, a section of social links 94 is provided, where the user can indicate various social media accounts to which the user belongs.

Referring to FIG. 13, the “posts” section 88 is shown. The “posts” section 88 comprises a collage of images from the user's previous posts, including media from emotional support messages that have been communicated to other users. In addition to emotional support messages, the content of the previous posts may include images and video captured by the user for announcing the user's mood, or media that was posted to other social media. Various images in the “posts” section 88 may be made larger, relative to other images, depending on a selection by the user, or according to a predetermined algorithm, such as the date the images were shared, or other factors. The “progress” section 90 of the editable user profile page 84 preferably links back to the “friends” screen 16 including the pending friends requests 22 screen, which enables the user to see the progress the user has made in addressing the emotional states of other users.

Referring to FIG. 14, in addition to the ability of a user to select the energy (both in terms of intensity and the positive/negative nature) of a user's mood, the application also includes functionality enabling a user to adjust the sensitivity regarding when the user is prompted to provide emotional support to “friend” accounts. This is done by adjusting a first “adjust sensitivity” slider 96 and a second “adjust sensitivity” slider 98 corresponding to the upper “energy” slider 28 and lower “energy” slider 30 (FIG. 9) indicators. Alternatively, the first “adjust sensitivity” slider 96 and the second “adjust sensitivity” slider 98 may correspond to an aggregate measurement of the upper “energy” slider 28 and the lower “energy” slider 30 as modified by the selections of the “Can you tell us more?” prompts 32.

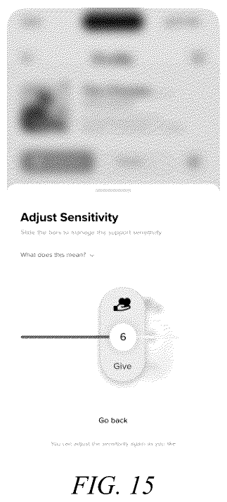

Referring to FIG. 15, an alternative “adjust sensitivity” slider is shown, wherein a single point of sensitivity is measured along a continuum, and can be set by the user according to preference.

What is claimed is:

1. A method for creating a virtual emotional social media support network for improving the mental health of users, the method comprising:
receiving, using a server processor, information associated with a first user of a first user device from a plurality of data sources, the data sources comprising:
the user device, the user device comprising a display mechanism and an input mechanism;
a user profile; and
a user-specific database;
identifying the optimal way to support the emotional health of a second user based on the received information associated with the first user;
determining when the second user may need emotional support, based on an algorithm accessible by the server processor, in conjunction with the received information associated with the first user of the first user device;
selecting at least one suggested action as determined by the algorithm, wherein the at least one suggested action is identified based on a plurality of settings associated with the first user profile; and
causing the at least one suggested action to be presented on a second user device of the second user.

2. The method of claim 1, wherein the data source further comprises environmental data of the first user, the environmental data comprising location, weather, or temperature.

3. The method of claim 1, wherein the data source further comprises health data of the first user, the health data comprising heart rate, body temperature, and nutrition.

4. The method of claim 1, wherein the data source further comprises behavioral data of the first user, the behavioral data comprising applications used, screen time, and purchases.

5. The method of claim 1, further comprising using a continuously updated algorithm based on the received information associated with the first user, such that the suggested actions are further optimized to meet the second user's emotional needs.

6. A system for creating a virtual emotional social media support network, the system comprising:
a server processor programmed to:
receive information associated with a first user of a first user device from a plurality of data sources, the data sources comprising:
the user device, the user device comprising a display mechanism and an input mechanism;
a user profile associated with the first user; and
a user-specific database associated with the first user;
identify and optimize a type and quantity of emotionally supportive information configured to support the emotional health of a second user based on the received information associated with the first user;
provide an algorithm accessible by the server processor, the algorithm configured to determine when the second user may need emotional support in conjunction with the received information associated with the first user device of the first user;
the algorithm configured to select at least one suggested action, wherein the at least one suggested action is identified based on a plurality of user settings associated with the user profile of the first user; and
cause the at least one suggested action to be presented on a second user device of the second user.

7. The system of claim 6, wherein the data source further comprises environmental data of the first user, the environmental data comprising location, weather, or temperature.

8. The system of claim 6, wherein the data source further comprises health data of the first user, the health data comprising heart rate, body temperature, and nutrition.

9. The system of claim 6, wherein the data source further comprises behavioral data of the first user, the behavioral data comprising applications used, screen time, and purchases.

10. The system of claim 6, further comprising a continuously updated algorithm based on the received information associated with the first user, such that the suggested actions are further optimized to meet the second user's emotional needs.

11. A non-transitory computer-readable medium containing computer executable instructions that, when executed by a processor, cause the processor to perform a method for creating a virtual emotional social media support network for a first user and a second user, the method comprising:
receiving, using a server processor, information associated with a user device of the first user from a plurality of data sources, the data sources comprising:
the user device, a display mechanism, and an input mechanism;
a first user profile; and
a first user-specific database;
identifying and optimizing a content provided by the user device, said content optimized to support the emotional health of the second user based on the received information associated with the second user;
determining when the second user may need emotional support, based on an algorithm accessible by the server processor, in conjunction with the received information associated with the user device of the first user;
selecting at least one suggested action as determined by the algorithm, wherein the at least one suggested action is identified based on a plurality of first user settings associated with a profile of the first user; and
causing the at least one suggested action to be presented on a second user device of the second user.

12. The non-transitory computer-readable medium of claim 11, wherein the data source further comprises environmental data of the first user, the environmental data comprising location, weather, or temperature.

13. The non-transitory computer-readable medium of claim 11, wherein the data source further comprises health data of the first user, the health data comprising heart rate, body temperature, and nutrition.

14. The non-transitory computer-readable medium of claim 11, wherein the data source further comprises behavioral data of the first user, the behavioral data comprising applications used, screen time, and purchases.

15. The non-transitory computer-readable medium of claim 11, further comprises a continuously updated algorithm based on the received information associated with the first user, such that the suggested actions are further optimized to meet the second user's emotional needs.
